(19) United States
(12) Patent Application Publication
(10) Pub. No.: US 2009/0264408 A1
(43) Pub. Date: Oct. 22, 2009

(54) EXTENDED RELEASE DOSAGE FORMS OF QUETIAPINE

(76) Inventors: Inder Gulati, New Delhi (IN); Rajan Kumar Verma, New Delhi (IN); Rajeev Singh Raghuvanshi, Gurgaon (IN); Romi Barat Singh, Varanasi (IN); Kumaravel Vivek, Chennai (IN); Sweta Varshney, Ghaziabad (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 12/420,504

(22) Filed: Apr. 8, 2009

(30) Foreign Application Priority Data
Apr. 17, 2008 (IN) 999/DEL/2008

(51) Int. Cl. A61K 31/554; A61P 25/18

(52) U.S. Cl. 514/211.13

(57) ABSTRACT

The present invention relates to an extended release dosage form of quetiapine wherein the dosage form comprises quetiapine and rate-controlling polymer selected from polyethylene oxide, sodium alginate and natural gum and combinations thereof. The dosage form may additionally comprise at least one water-insoluble polymer.

### TECHNICAL FIELD OF THE INVENTION

The present invention relates to extended release dosage forms for oral administration comprising quetiapine and processes for the preparation thereof.

### BACKGROUND OF THE INVENTION

Quetiapine is a psychotropic drug belonging to a chemical class, the dibenzothiazepine derivatives and is chemically designated as 11-[4-[2-(2-hydroxyethoxy)ethyl]-1-piperazinyl]dibenzo[b,f][1,4]thiazepine. Quetiapine acts as an antagonist at several neurotransmitter receptors including dopamine D₁ and D₂ receptors, serotonin 5HTA₁ and 5HT₂ receptors, histamine H₁ receptor and adrenergic α₁ and α₂ receptors. Quetiapine is thought to exert its antipsychotic effects primarily via antagonism of dopamine D₂ receptor and serotonin 5HT₂ receptors.

Currently, quetiapine is commercially available as conventional immediate release tablets in 25, 50, 100, 200, 300 and 400 mg strengths marketed by Astra Zeneca, under the brand name Seroquel®, requiring two or three times a day dosing. It is also available as extended release tablets in 50, 150, 200, 300 and 400 mg strengths under the brand name Seroquel® XR. These tablets contain quetiapine as a hemifumarate salt, lactose monohydrate, microcrystalline cellulose, sodium citrate, hypromellose, magnesium stearate and hypromellose, polyethylene glycol 400, titanium dioxide, yellow iron oxide (200 and 300 mg tablets) in the film-coat.

It is desirable in the therapeutic and prophylactic treatment of diseases to provide the active pharmaceutical ingredient in extended release form. Extended release dosage forms can increase patient compliance due to reduction in frequency of dosing. They may also reduce the severity and frequency of side effects, as they typically maintain substantially constant plasma levels. This is especially important in the treatment of schizophrenia and bipolar mania, for the alleviation of psychosis, where blood levels of medicament are desirably maintained at a therapeutically effective level to provide symptomatic relief.

Quetiapine and its pharmaceutically acceptable salts, its preparation, physical properties and beneficial pharmacological properties are disclosed in U.S. Pat. No. 4,879,288 and EP Patent Nos. 240,228 and 282,236.

WO 2007/000778 exemplifies modified release matrix tablets comprising quetiapine fumarate, a polymer system in an amount of less than about 80% w/w of the composition comprising at least two swellable pH independent polymers wherein at least one is hydrophilic, and additionally, at least one pH dependent hydrophilic release controlling polymer; and other pharmaceutically acceptable excipients.

WO 2007/110878 exemplifies hard gelatin capsules containing sustained release granules comprising quetiapine fumarate, at least one solubilizer (e.g., propylene glycol caprylate/caprate, Labrafac™), a release rate-controlling polymer system comprising hydrophilic polyethylene oxide and hydroxyethylcellulose, and other pharmaceutically acceptable excipients.

WO 2007/086079 discloses once a day sustained release matrix tablets comprising phenothiazine derivative, a channelizer, a rate-controlling agent and suitable pharmaceutical excipients. Examples cited therein disclose sustained release formulations of quetiapine fumarate with water-soluble polymer i.e. hydroxypropyl methylcellulose, and other excipients.

US Publication No. 2005/0158383 discloses sustained release dosage forms of quetiapine in a waxy matrix. In the formulations described therein, the coating composition comprising a hydrophilic polymer may be press coated onto the core.

WO 01/21179 discloses a granule formulation comprising quetiapine or a pharmaceutically acceptable salt thereof and freely or very water-soluble binder.

WO 03/39516 discloses a method for improving dissolution of poorly dispersible medicament like quetiapine, which comprises mixing the poorly dispersible medicament with a floating agent and/or a surfactant and granulating the mixture.

A typical sustained release formulation of quetiapine is described in U.S. Pat. No. 5,948,437. It discloses matrix formulations of quetiapine wherein the matrix comprises gelling agents, particularly, hydroxypropyl methylcellulose for sustained release. The patent further discloses that it is difficult to formulate sustained release formulations of soluble medicaments like quetiapine fumarate and gelling agents like hydroxypropyl methylcellulose for reasons of dose dumping. That is, release of the active ingredient is delayed for a time but once the release begins to occur, the rate of release is very high. Further some degree of diurnal variation in plasma concentration of the active ingredient has also been observed and lastly, it has been found to be difficult to achieve the desired dissolution profiles or to control the rate of release of the soluble medicament. The sustained release tablets of quetiapine have been prepared with hydroxypropyl methylcellulose as the sole rate-controlling polymer.

### SUMMARY OF THE INVENTION

Extended release dosage forms of quetiapine are disclosed herein, which would provide the desired pharmacokinetic profile wherein the dosage forms comprise a matrix containing quetiapine and a rate-controlling polymer and one or more of pharmaceutically acceptable excipients as well as process for the preparation thereof. The rate-controlling polymer may be a polymer selected from polyethylene oxide, sodium alginate and natural gums such as xanthan gum or locusts gum. It may additionally comprise at least one water-insoluble polymer.

In one general aspect, extended release dosage forms of quetiapine are disclosed herein, wherein the dosage forms comprise quetiapine and a rate-controlling polymer such as, for example, polyethylene oxide, sodium alginate and natural gum and combinations thereof.

In another general aspect, extended release dosage forms of quetiapine are disclosed herein, wherein the dosage forms comprise quetiapine and a rate-controlling polymer such as, for example, polyethylene oxide, sodium alginate and natural gum and combinations thereof; wherein the dosage form provides therapeutically effective plasma levels of quetiapine for a period of up to about 24 hours.

In another general aspect, processes for preparing extended release dosage forms of quetiapine are provided herein wherein the process comprises mixing quetiapine and a rate-controlling polymer such as, for example, polyethylene oxide, sodium alginate and natural gum and combinations thereof, and processing into solid dosage forms.

In another general aspect, processes for preparing extended release dosage forms of quetiapine are provided herein wherein the processes comprise mixing quetiapine and a rate-controlling polymer such as, for example, polyethylene oxide, sodium alginate and natural gum and combinations thereof, and one or more other pharmaceutical excipients; granulating the blend with a granulating liquid; drying the granules; lubricating the granules with a lubricant; and compressing the granules into a tablet.

In another general aspect, extended release dosage forms of quetiapine are provided herein, wherein the dosage forms comprise quetiapine and a rate-controlling polymer selected from polyethylene oxide, sodium alginate and natural gum and combinations thereof, and at least one water-insoluble polymer.

In another general aspect, extended release dosage forms of quetiapine are provided herein, wherein the dosage forms comprise quetiapine and a rate-controlling polymer selected from, for example, polyethylene oxide, sodium alginate and natural gum and combinations thereof, and at least one water-insoluble polymer, wherein the dosage forms provide therapeutically effective plasma levels of quetiapine for a period of up to about 24 hours.

In another general aspect, extended release dosage forms of quetiapine are provided herein, wherein the dosage forms comprise quetiapine and a rate-controlling polymer selected from, for example, polyethylene oxide, sodium alginate and natural gum and combinations thereof, and at least one water-insoluble polymer, wherein the water-insoluble polymer is selected from ammonio-methacrylate copolymers, methacrylic acid copolymers, ethyl cellulose and combinations thereof.

In another general aspect, processes for preparing extended release dosage forms of quetiapine are provided herein, wherein the processes comprise mixing quetiapine and a rate-controlling polymer selected from, for example, polyethylene oxide, sodium alginate and natural gum and combinations thereof, and at least one water-insoluble polymer and processing into solid dosage forms.

In another general aspect, processes for preparing extended release dosage forms of quetiapine are provided herein, wherein the processes comprise mixing quetiapine and a rate-controlling polymer selected from, for example, polyethylene oxide, sodium alginate and natural gum and combinations thereof, and one or more of other pharmaceutical excipients; granulating the blend with a solution/dispersion of water-insoluble polymer; drying the granules; lubricating the granules with a lubricant; and compressing the granules into a tablet.

In another general aspect, processes for preparing extended release dosage forms of quetiapine are provided herein, wherein the processes comprise mixing quetiapine and one or more of other pharmaceutical excipients; granulating the blend with a solution/dispersion of water-insoluble polymer; mixing the granules with a rate-controlling polymer selected from, for example, polyethylene oxide, sodium alginate and natural gum and combinations thereof and one or more of other pharmaceutical excipients; and compressing the resultant blend into a tablet.

### DETAILED DESCRIPTION OF THE INVENTION

“Quetiapine”, as recited herein means quetiapine or a pharmaceutically acceptable form of quetiapine, including without limitation, its free base form, and all pharmaceutically acceptable salts, complexes, enantiomer, solvates, hydrates, and polymorphs. The preferred quetiapine salt is quetiapine hemifumarate.

The rate-controlling polymer may be, for example, polyethylene oxide, sodium alginate or natural gums. Polyethylene oxide may be of different viscosity grades such as Polyox® WSR 303, Polyox® WSR 301, Polyox® WSR N-60K, or Polyox® WSR Coagulant available from Colorcon. Natural gums may be, for example, gum tragacanth, locust bean gum, guar gum, karaya gum or xanthan gum. The dosage forms as described herein may additionally comprise at least one water-insoluble polymer. Examples of suitable water-insoluble polymers include acrylates such as methacrylates, polymethacrylic acid-based polymers and copolymers such as those sold under the trade name Eudragit®; cellulose derivatives such as ethyl cellulose, cellulose acetate, cellulose acylate, cellulose diacylate, cellulose triacylate, cellulose diacetate, cellulose triacetate, mono-, di- and tri-cellulose alkanylates, mono-, di-, and tri-cellulose arylates, mono-, di- and tri-cellulose adenylates; polyethylene; polyvinyl chloride; vinyl acetate/vinyl chloride copolymer; vinylidene chloride/acrylonitrile copolymer; high molecular weight polyvinylalcohols and mixtures thereof. Particular embodiments include Eudragit® RL/RS and ethyl cellulose. The total amount of rate-controlling polymers in the tablet relative to quetiapine depends upon the rate of drug release desired and also upon the type and molecular weight of the polymers and other excipients present in the formulation and may vary from about 5% to about 95% by weight of the composition. The dosage forms may also comprise other rate-controlling polymers such as crosslinked polyacrylic acids (Carbopols), polyvinylpyrrolidone, and the like.

The term “dosage form” as recited herein includes dosage forms such as tablets, granules, and capsules filled with granules or tablets.

The other pharmaceutical excipients may be one or more diluents, binders, pH modifiers, anti-oxidants, disintegrants, glidants/lubricants and plasticizers.

Suitable diluents may be, for example, one or more conventional diluents such as microcrystalline cellulose, silicified microcrystalline cellulose, lactose, mannitol, sorbitol, calcium phosphate, calcium sulfate, calcium carbonate, starch, starch pregelatinized, and the like.

Suitable binders may be, for example, one or more polyvinylpyrrolidone, cross-linked polyvinylpyrrolidone, polyvinyl alcohol, carboxymethyl cellulose, hydroxypropyl methylcellulose, hydroxypropyl cellulose, microcrystalline cellulose, silicified microcrystalline cellulose, starch pregelatinized, and the like. Suitable pH modifiers may be, for example, benzoic acid, citric acid, tartaric acid or metal salts thereof. Suitable anti-oxidants may be selected from butylated hydroxytoluene, butylated hydroxyanisole, Vitamin E, tocopherol, and the like. Particular embodiments include butylated hydroxytoluene. Suitable disintegrants may be, for example, carboxymethyl cellulose, sodium carboxymethyl cellulose, cross-linked sodium carboxymethyl cellulose, sodium starch glycolate, starch, pregelatinized starch, hydroxypropyl starch, and the like. Suitable glidants/lubricants may include one or more of magnesium stearate, calcium stearate, zinc stearate, stearic acid, talc, colloidal silicon dioxide, hydrogenated vegetable oil, polyethylene glycol, sodium stearyl fumarate, and the like.

Suitable granulating liquid may be a solvent such as water, isopropyl alcohol, acetone, methanol, ethanol, dichloromethane or mixtures thereof or a solution/dispersion of a polymer, for example, polyvinylpyrrolidone.

The rate-controlling polymers may be provided as solutions/dispersions in organic solvent/water or mixture of organic solvent and water and may comprise suitable plasticizers. Examples of plasticizers include citrate esters, phthalate esters, triacetin, castor oil, polyethylene glycols, propylene glycol, and the like.

Tablets can additionally be coated with non-rate-controlling polymer compositions like Opadry® sold by Colorcon to impart aesthetic appeal.

The compositions described herein may be prepared by conventional processes using commonly available equipment. The process may involve wet granulation, dry granulation or direct compression processes.

Particular pharmaceutical compositions of the present invention may take the form of several different embodiments. In one embodiment, the extended release dosage form is a tablet comprising quetiapine and a rate-controlling polymer such as, for example, polyethylene oxide, sodium alginate and natural gums or combinations thereof and one or more of other pharmaceutical excipients. In another embodiment, the extended release dosage form is a tablet comprising quetiapine and a rate-controlling polymer such as, for example, polyethylene oxide, sodium alginate and natural gums or combinations thereof; at least one pH modifier; and one or more other pharmaceutical excipients.

In another embodiment, the extended release dosage form is a tablet, prepared by a process comprising mixing quetiapine, diluents, pH modifier, rate-controlling polymer; granulating with a granulating liquid; drying the granules; mixing the dried granules with lubricant and glidant and compressing into tablet using appropriate tooling. In another embodiment, the extended release dosage form is a tablet comprising quetiapine and a rate-controlling polymer selected from polyethylene oxide, sodium alginate and natural gums or combination thereof and at least one water-insoluble polymer and one or more other pharmaceutical excipients. In the above embodiment, the water-insoluble polymer may be selected from ethyl cellulose or Eudragit®.

In another embodiment, the extended release dosage form is a tablet and is prepared by the process comprising mixing quetiapine and a rate-controlling polymer selected from polyethylene oxide, sodium alginate and natural gums and combinations thereof and one or more of other pharmaceutical excipients; granulating with a solution/dispersion of at least one water-insoluble polymer; drying the granules; mixing the dried granules with lubricant and glidant and compressing into tablet using appropriate tooling. In another embodiment, the extended release tablet is prepared by the process comprising mixing quetiapine and one or more of other pharmaceutical excipients; granulating the blend with a solution/dispersion of water-insoluble polymer; mixing the granules with a rate-controlling polymer selected from polyethylene oxide, sodium alginate and natural gum and combinations thereof and one or more of other pharmaceutical excipients; and compressing the resultant blend into a tablet.

The following examples are given for purpose of illustrating the present invention and do not limit the scope of the invention in any way.

### Example 1 (1a-1c)

### Extended Release Tablets of Quetiapine Comprising Polyox as the Rate-Controlling Polymer

| | | Quantity (mg) | | |
|---|---|---|---|---|
| S. No. | Ingredient | Example 1a | Example 1b | Example 1c |
| 1 | Quetiapine fumarate | 461.0 | 233.1 | 230 |
| 2. | Lactose | 39.5 | 37.1 | 53.8 |
| | monohydrate | | | |
| 3. | Microcrystalline | 39.5 | 37.1 | 53.8 |
| | cellulose | | | |
| 4. | Sodium citrate | 100.0 | 62.5 | 137.5 |
| 5 | Polyox ® WSR 303 | 250.0 | — | — |
| 6. | Polyox ® WSR | — | 192.5 | 154.0 |
| | N-60K | | | |
| 7 | Polyox ® WSR 301 | — | — | 38.5 |
| 8. | Magnesium stearate | 15.0 | 11.0 | 11.0 |
| 9. | Colloidal silicon | 2.5 | 1.5 | 1.5 |
| | dioxide | | | |
| 10. | Butylated | — | 0.2 | — |
| | hydroxytoluene | | | |
| | Tablet Weight | 907.5 | 575.0 | 680.1 |
| 11. | Opadry ® Coating | 22.5 | — | — |
| | Final Tablet Weight | 930.0 | — | — |

### Procedure for Example 1:

1. Accurately weighted quantities of quetiapine fumarate, lactose monohydrate, microcrystalline cellulose, sodium citrate and polyox were mixed in a suitable blender.
2. The above blend was transferred to rapid mixer granulator and granulated with isopropyl alcohol and purified water.
3. The granules were dried in a fluidized bed dryer.
4. The dried granules were mixed with magnesium stearate and colloidal silicon dioxide and butylated hydroxytoluene (if present).
5. The blend of step 4 was compressed into tablets using appropriate tooling and the resultant tablets were optionally coated with Opadry®.

TABLE 1
Dissolution profile of tablets prepared as per Examples 1a-1c in
900 mL of 0.1N HCl in USP Dissolution Apparatus Type I at 100 rpm

| | % of Drug Released | | |
|---|---|---|---|
| Time (h) | Example 1a | Example 1b | Example 1c |
| 0 | 0 | 0 | 0 |
| 1 | 15 | 20 | 16 |
| 2 | 26 | 33 | 27 |
| 4 | 46 | 57 | 48 |
| 6 | 65 | 78 | 70 |
| 8 | 82 | 93 | 86 |
| 12 | 98 | 105 | 98 |
| 16 | 99 | — | — |
| 20 | 99 | — | — |

### Example 2

### Extended Release Tablets of Quetiapine Comprising Polyox and Xanthan Gum as the Rate-Controlling Polymers

| | | Quantity (mg) |
|---|---|---|
| S. No. | Ingredient | Example 2 |
| 1. | Quetiapine fumarate | 461.0 |
| 2. | Lactose monohydrate | 39.5 |
| 3. | Microcrystalline cellulose | 39.5 |
| 4. | Sodium citrate | 100.0 |
| 5. | Xanthan gum | 100.0 |
| 6. | Polyox ® | 150.0 |
| 7. | Magnesium stearate | 13.5 |
| 8. | Colloidal silicon dioxide | 2.5 |
| | Tablet Weight | 906.0 |
| 9. | Opadry ® coating | 24.0 |
| | Final Tablet Weight | 930.0 |

### Procedure for Example 2:

1. Accurately weighted quantities of quetiapine fumarate, lactose monohydrate, microcrystalline cellulose, sodium citrate, xanthan gum and polyethylene oxide were mixed in a suitable blender.
2. The above blend was transferred to rapid mixer granulator and granulated with isopropyl alcohol and purified water.
3. The granules were dried in a fluidized bed dryer.
4. The dried granules were mixed with magnesium stearate and colloidal silicon dioxide.
5. The blend of step 4 was compressed into tablets using appropriate tooling and the resultant tablets were coated with Opadry®.

TABLE 2
Dissolution profile of tablets prepared as per Example 2 in
900 mL of 0.1N HCl in USP dissolution Apparatus Type I at 100 rpm

| | % of Drug Released |
|---|---|
| Time (h) | Example 2 |
| 0 | 0 |
| 1 | 15 |
| 2 | 24 |
| 4 | 39 |
| 6 | 50 |
| 8 | 60 |
| 12 | 76 |
| 16 | 86 |
| 20 | 90 |

### Example 3

### Extended Release Tablets of Quetiapine Comprising Sodium Alginate as the Rate-Controlling Polymer

| | | Quantity (mg) |
|---|---|---|
| S. No. | Ingredient | Example 3 |
| 1. | Quetiapine fumarate | 461.0 |
| 2. | Lactose monohydrate | 39.5 |
| 3. | Microcrystalline cellulose | 39.5 |
| 4. | Sodium citrate | 100.0 |
| 5. | Sodium alginate | 250.0 |
| 6. | Magnesium stearate | 13.5 |
| 7. | Colloidal silicon dioxide | 2.5 |
| | Tablet Weight | 906.0 |
| 8. | Opadry ® Coating | 24.0 |
| | Final Tablet Weight | 930.0 |

### Procedure for Example 3:

1. Accurately weighted quantities of quetiapine fumarate, lactose monohydrate, microcrystalline cellulose, sodium citrate and sodium alginate were mixed in a suitable blender.
2. The above blend was transferred to rapid mixer granulator and granulated with purified water.
3. The granules were dried in a fluidized bed dryer.
4. The dried granules were mixed with magnesium stearate and colloidal silicon dioxide.
5. The blend of step 4 was compressed into tablets using appropriate tooling and the resultant tablets were coated with Opadry®.

TABLE 3
Dissolution profile of tablets prepared as per Example 3 in
900 mL of 0.1N HCl in USP Dissolution Apparatus Type I at 100 rpm

| | % of Drug Released |
|---|---|
| Time (h) | Example 3 |
| 0 | 0 |
| 1 | 22 |
| 2 | 34 |
| 4 | 52 |
| 6 | 65 |
| 8 | 76 |
| 12 | 90 |
| 16 | 97 |
| 20 | 99 |

### Example 4 (4a-4b)

### Extended Release Tablets of Quetiapine Comprising Polyox and Ethyl Cellulose as the Rate-Controlling Polymers

| | | Quantity (in mg) | |
|---|---|---|---|
| S. No. | Ingredient | Example 4a | Example 4b |
| 1. | Quetiapine fumarate | 230.3 | 234.6 |
| 2. | Lactose monohydrate | 53.6 | 51.5 |
| 3. | Microcrystalline cellulose | 53.6 | 51.5 |
| 4. | Sodium citrate | 62.5 | 62.5 |
| 5. | Polyox ® WSR N-60K | 137.5 | 137.5 |
| 6. | Ethyl cellulose | 13.2 | 5.0 |
| 7. | Magnesium stearate | 11.0 | 11.0 |
| 8. | Colloidal silicon dioxide | 1.5 | 1.5 |
| 9. | Butylated hydroxytoluene | — | 0.2 |
| | Tablet Weight | 563.2 | 555.3 |

### Procedure for Example 4:

1. Accurately weighted quantities of quetiapine fumarate, lactose monohydrate, microcrystalline cellulose, sodium citrate and Polyox® were mixed in a suitable blender.
2. The above blend was transferred to a rapid mixer granulator and granulated with ethyl cellulose binder solution in isopropyl alcohol and dichloromethane.
3. The granules were dried in a fluidized bed dryer.
4. The dried granules were mixed with butylated hydroxytoluene (if present), magnesium stearate and colloidal silicon dioxide.
5. The blend of Step 4 was compressed into tablets using appropriate tooling.

TABLE 4
Dissolution profile of tablets prepared as per Examples 4a-4b
in 900 mL of 0.1N HCl in USP Dissolution Apparatus Type I at 100 rpm

% Drug Released

| Time (h) | Example 4a | Example 4b |
|---|---|---|
| 0 | 0 | 0 |
| 1 | 19 | 22 |
| 2 | 32 | 37 |
| 4 | 55 | 61 |
| 6 | 74 | 81 |
| 8 | 88 | 94 |
| 12 | 97 | 104 |

### Example 5 (5a-5d)

### Extended Release Tablets of Quetiapine Comprising Polyox and Eudragit® as Rate-Controlling Polymers

| | | Quantity (in mg) | | | |
|---|---|---|---|---|---|
| | | Example | Example | Example | |
| S. No. | Ingredients | 5a | 5b | 5c | Example 5d |
| 1. | Quetiapine fumarate | 234.6 | 234.6 | 233.4 | 233.4 |
| 2. | Eudragit ® RS30D | 45.4 | 45.4 | 22.0 | 22.0 |
| 3. | Triethyl citrate | 6.8 | 6.8 | 3.3 | 3.3 |
| 4. | Talc | 18.1 | 18.1 | 8.8 | 8.8 |
| 5. | Purified water | q.s. | q.s. | q.s. | q.s. |
| 6. | Colloidal silicon dioxide | 0.8 | 0.8 | 0.8 | 0.8 |
| 7. | Lactose monohydrate | 54.5 | 54.5 | 54.5 | 54.5 |
| 8. | Microcrystalline cellulose | 54.5 | 54.5 | 54.5 | 54.5 |
| 9. | Sodium citrate | 75.0 | 75.0 | 75.0 | 75.0 |
| 10. | Polyox ® WSR N-60K | 100.0 | 75.0 | 135.0 | 85.0 |
| 11. | Magnesium stearate | 12.0 | 12.0 | 12.0 | 12.0 |
| 12. | Colloidal silicon dioxide | 1.5 | 1.5 | 1.5 | 1.5 |
| 13. | Butylated hydroxytoluene | 0.2 | 0.2 | 0.2 | 0.2 |
| | Total Tablet Weight | 603.5 | 578.4 | 601.0 | 551.0 |

### Procedure for Example 5:

1. Quetiapine fumarate and colloidal silicon dioxide were mixed together and loaded in Glatt.
2. Dispersion of talc was prepared in water and triethyl citrate was added to it and stirred.
3. Dispersion of step 2 was added in Eudragit® dispersion and stirred for 45 minutes.
4. Blend of step 1 was granulated with dispersion of step 3.
5. The resultant granules were mixed with lactose monohydrate, microcrystalline cellulose and Polyox®.
6. Blend of step 5 was mixed with butylated hydroxytoluene, magnesium stearate and colloidal silicon dioxide.
7. The above blend was compressed into tablets using appropriate tooling.

TABLE 5
Dissolution profile of tablets prepared as per Examples 5a-5d
in 900 ml of 0.1N HCl in USP Dissolution Apparatus Type I at 100 rpm

% Drug Released

| | Example | Example | Example | Example |
|---|---|---|---|---|
| Time (h) | 5a | 5b | 5c | 5d |
| 0 | 0 | 0 | 0 | 0 |
| 1 | 19 | 22 | 22 | 23 |
| 2 | 33 | 36 | 36 | 39 |
| 4 | 53 | 57 | 56 | 62 |
| 6 | 69 | 74 | 73 | 79 |
| 8 | 83 | 89 | 87 | 95 |
| 12 | 99 | 103 | 97 | 104 |

### Example 6 (6a-6d)

### Extended Release Tablets of Quetiapine Comprising Polyox and Eudragit as the Rate-Controlling Polymers

| | | Quantity (in mg) | | | |
|---|---|---|---|---|---|
| | | Example | Example | Example | |
| S. No. | Ingredients | 6a | 6b | 6c | Example 6d |
| 1. | Quetiapine fumarate | 233.4 | 233.4 | 232.1 | 232.1 |
| 2. | Eudragit ® RS30D | 22.0 | 22.0 | 56.0 | 56.0 |
| 3. | Triethyl citrate | 3.3 | 3.3 | 8.4 | 8.4 |
| 4. | Talc | 8.8 | 8.8 | 22.4 | 22.4 |
| 5. | Purified water | q.s. | q.s. | q.s. | q.s. |
| 6. | Colloidal silicon dioxide | 0.8 | 0.8 | 0.8 | 0.8 |
| 7. | Lactose monohydrate | 54.5 | 54.5 | 54.5 | 54.5 |
| 8. | Microcrystalline cellulose | 54.5 | 54.5 | 54.5 | 54.5 |
| 9. | Sodium citrate | 75.0 | 75.0 | 75.0 | 75.0 |
| 10. | Polyox ® WSR 303 | 85.0 | 135.0 | — | — |
| 11. | Polyox ® WSR 301 | — | — | 100.0 | 135.0 |
| 12. | Magnesium stearate | 12.0 | 12.0 | 12.0 | 12.0 |
| 13. | Colloidal silicon dioxide | 1.5 | 1.5 | 1.5 | 1.5 |
| 14. | Butylated hydroxytoluene | 0.2 | 0.2 | 0.2 | 0.2 |
| | Total Tablet Weight | 551.0 | 601.0 | 617.4 | 652.4 |

### Procedure for Example 6:

Same as described above for Example 5.

TABLE 6
Dissolution profile of tablets prepared as per Examples 6a-6d
in 900 ml of pH 6.8 phosphate buffer in USP Dissolution
Apparatus Type I at 100 rpm

% Drug Released

| | Example | Example | Example | Example |
|---|---|---|---|---|
| Time (h) | 6a | 6b | 6c | 6d |
| 0 | 0 | 0 | 0 | 0 |
| 1 | 3 | 2 | 3 | 2 |
| 2 | 5 | 4 | 6 | 5 |
| 4 | 9 | 7 | 13 | 12 |
| 8 | 17 | 14 | 28 | 27 |
| 12 | 26 | 21 | 45 | 47 |
| 16 | 34 | 30 | 59 | 61 |
| 20 | 42 | 38 | 66 | 69 |
| 24 | 51 | 46 | 72 | 74 |

### Example 7 (7a-7b)

### Extended Release Tablets of Quetiapine Comprising Polyox, Xanthan Gum and Eudragit® as the Rate-Controlling Polymers

| | | Quantity (mg) | |
|---|---|---|---|
| S. No. | Ingredients | Example 7a | Example 7b |
| 1. | Quetiapine fumarate | 233.4 | 233.4 |
| 2. | Eudragit ® RS 30D | 22.0 | 22.0 |
| 3. | Triethyl citrate | 3.3 | 3.3 |
| 4. | Talc | 8.8 | 8.8 |
| 5. | Purified water | q.s. | q.s. |
| 6. | Colloidal silicon dioxide | 0.8 | 0.8 |
| 7. | Lactose monohydrate | 54.5 | 54.5 |
| 8. | Microcrystalline cellulose | 54.5 | 54.5 |
| 9. | Sodium citrate | 75.0 | 75.0 |
| 10. | Polyox ® WSR N-60K | 115.0 | 75.0 |
| 11. | Xanthan gum | 25.0 | 10.0 |
| 12. | Magnesium stearate | 12.0 | 12.0 |
| 13. | Colloidal silicon dioxide | 1.5 | 1.5 |
| 14. | Butylated hydroxytoluene | 0.2 | 0.2 |
| | Total Tablet Weight | 606.0 | 550.9 |

### Procedure for Example 7:

1. Quetiapine fumarate and colloidal silicon dioxide were mixed together and loaded in Glatt.
2. Dispersion of talc was prepared in water and to it triethyl citrate was added.
3. Dispersion of step 2 was added in Eudragit® dispersion and stirred for 45 min.
4. Blend of step 1 was granulated with dispersion of step 3.
5. The resultant granules were mixed with lactose monohydrate, microcrystalline cellulose, Polyox® and xanthan gum.
6. Blend of step 5 was mixed with butylated hydroxytoluene, magnesium stearate and colloidal silicon dioxide.
7. The above blend was compressed into tablets using appropriate tooling.

TABLE 7
Dissolution profile of tablets prepared as per Examples 7a-7b
in 900 ml of 0.1N HCl in USP Dissolution Apparatus Type I at 100 rpm

% of Drug Released

| Time (h) | Example 7a | Example 7b |
|---|---|---|
| 0 | 0 | 0 |
| 1 | 23 | 24 |
| 2 | 41 | 39 |
| 4 | 57 | 62 |
| 6 | 73 | 82 |
| 8 | 84 | 95 |
| 12 | 97 | 103 |

We claim:

1. An extended release dosage form comprising quetiapine and a rate-controlling polymer selected from polyethylene oxide, sodium alginate and natural gum and combinations thereof and other pharmaceutically acceptable excipients.

2. The extended release dosage form according to claim 1, wherein other pharmaceutically acceptable excipients comprise one or more of plasticizers, solvents, binders, diluents, disintegrants, pH modifiers, antioxidants, lubricants, glidants or mixtures thereof.

3. A process for the preparation of extended release dosage form according to claim 1, wherein the process comprises mixing quetiapine and a rate-controlling polymer selected from polyethylene oxide, sodium alginate and natural gum and combinations thereof and compressing the blend into a tablet.

4. A process for the preparation of extended release dosage form according to claim 1, wherein the process comprises mixing quetiapine and a rate-controlling polymer selected from polyethylene oxide, sodium alginate and natural gum and combinations thereof and one or more of other pharmaceutically acceptable excipients; granulating the blend with a granulating liquid; drying the granules; lubricating the granules with a lubricant; and compressing the granules into a tablet.

5. The extended release dosage form according to claim 1, further comprising at least one water-insoluble polymer.

6. The extended release dosage form according to claim 5, wherein the water-insoluble polymer is selected from ammonio-methacrylate copolymers, methacrylic acid copolymers, ethyl cellulose, and combinations thereof.

7. A process for the preparation of extended release dosage form according to claim 5, wherein the process comprises mixing quetiapine and a rate-controlling polymer selected from polyethylene oxide, sodium alginate and natural gum and combinations thereof and at least one water-insoluble polymer and compressing the blend into a tablet.

8. A process for the preparation of extended release dosage form according to claim 5, wherein the process comprises mixing quetiapine and a rate-controlling polymer selected from polyethylene oxide, sodium alginate and natural gum and combinations thereof and one or more of other pharmaceutically acceptable excipients; granulating the blend with a solution/dispersion of water-insoluble polymer; drying the granules; lubricating the granules with a lubricant; and compressing the granules into a tablet.

9. A process for the preparation of extended release dosage form according to claim 5, wherein the process comprises mixing quetiapine and one or more of other pharmaceutically acceptable excipients; granulating the blend with a solution/dispersion of water-insoluble polymer; mixing the granules with a rate-controlling polymer selected from polyethylene oxide, sodium alginate and natural gum and combinations thereof and one or more of other pharmaceutically acceptable excipients; and compressing the resultant blend into a tablet.

10. The extended release dosage form of quetiapine according to any of the preceding claims, wherein the dosage form exhibits the following in vitro dissolution profile, when measured in a USP dissolution apparatus type I, at 100 rpm, at a temperature of 37±0.5° C. in 900 ml of 0.1N hydrochloric acid;
at most about 50% of the drug is released in 2 hours;
at most about 75% of the drug is released in 4 hours and
at most about 99% of the drug is released in 8 hours.
